(19) United States
(12) Patent Application Publication
(10) Pub. No.: US 2008/0014123 A1
(43) Pub. Date: Jan. 17, 2008

(54) Container

(76) Inventor: Marko Voho, Vantaa (FI)

Correspondence Address: NIXON & VANDERHYE, PC, 901 NORTH GLEBE ROAD, 11TH FLOOR, ARLINGTON, VA, 22203, US

(21) Appl. No.: 11/597,631

(22) PCT Filed: May 26, 2005
(86) PCT No.: PCT/FI05/00242

§ 371 Date: Sep. 6, 2007

(30) Foreign Application Priority Data
May 27, 2004 (FI) 20040725

(51) Int. Cl. B01L 3/02; B01L 11/00; B01L 9/00; G01N 33/00; B01L 3/00

(52) U.S. Cl. 422/100; 422/102; 422/104; 422/68.1; 422/99

(57) ABSTRACT

The invention relates to a container for a liquid sample, which container (1) comprises an elongate casing and into which container a liquid sample may be taken and in which it may be examined. The container (1) comprises an open lower end and an open upper end and the sample in the container may be examined in the longitudinal direction of the container, especially from above.

### TECHNICAL FIELD

The invention relates to the handling of liquid samples and to a container into which the liquid sample is taken and in which it is examined. The invention can be used for example for analysing samples in clinical laboratories.

### TECHNICAL BACKGROUND

Plates, in which a plurality of sample vessels, i.e. wells, have been combined, are often used for handling liquid samples in laboratories In this case the analyses can be conveniently performed in series. A standard plate that has already been used a long time is a so-called micro titration plate, which has 96 wells in a 8*12 matrix at 9 mm intervals. Later also plates, which have 384 or 1536 wells on a corresponding area have been taken into use. The samples are often analysed on the plates optically using equipment, in which light is lead to or from the sample. In this case for example in a fluorometric analysis excitation light is conducted to and emission light is conducted from the sample. Both lights can be conducted from above.

The smaller the sample wells are in size, the more difficult it is to dispense the samples into them. For example, in the above-mentioned plate having 1536 wells the inner diameter of the well is only approximately 2 mm.

### SUMMARY OF THE INVENTION

In accordance with the independent claims a container for a liquid sample and a method for analysing a sample have now been invented. The other claims describe some preferred embodiments of the invention.

The invention relates to a container, which comprises an open upper end and an open lower end, and into which a sample is taken and in which the sample is examined in the longitudinal direction of the container, especially from above, for example optically. The sample may be sucked into the container but it is not necessary to dispense the sample again to a separate vessel for analysis.

### DRAWINGS

The accompanying drawings form a part of the written disclosure of the invention and relate to the following detailed description of the invention. In the drawings:

FIGS. 1a and 1b illustrate a container placed in a suction device and in a rack

FIG. 2 illustrates a container to be placed in a suction device

FIG. 3 illustrate containers of FIG. 2 in a rack during analysis.

### DETAILED DESCRIPTION OF THE INVENTION

The container according to the invention comprises an open lower end and an open upper end. A sample taken into the container may be examined especially from above. The sample may be taken into the container especially from below. A suction device, such as a pipette, may be used for this purpose. The container may be detached from the suction device for examination.

The diameter of the lower end of the container is preferably so small that the sample remains in the container even when no suction is directed to the container from the upper end. The container may widen from the lower end toward the upper end.

An upwards widening container may comprise a narrower lower part and a wider upper part connected to it, so that a bottom reaching outwards from the boundary of the parts is formed on the upper part. The bottom of the upper part may be substantially horizontal. The bottom may be surrounded by an edge, especially by a substantially vertical edge. The sample may, be taken into the container in such an amount that its upper surface reaches the upper part.

The container may be placed to a rack for examination, in which rack there is one or especially several places for the container. In particular a rack, which corresponds to some standard well plate used in analyses, may be used. The rack may contain a cover that can be attached to the rack so that the containers remain closed inside. The cover may be such that it closes the upper ends of the containers. In this way the samples may also be stored and transported in the containers.

When necessary, the container can be agitated in order to mix the liquid in it and thus enhance the desired reaction, for example. The agitation may be performed to a container placed in a suction device or in a rack, for example.

The containers can be combined as a unit formed by-two or more, for example four containers. The container unit may have an edge surrounding the separate containers. The edge can be such that the container unit may be attached from it to a suction device, by which suction can be directed to the containers simultaneously. In this case the upper ends of the containers may comprise upper containers separated by lower separating walls.

The inner surface of the container may be coated with a suitable reagent, for example with a reagent responsive to the sample.

The sample can especially be analysed optically, for example fluorometrically, luminometrically, nefelometrically or microscopically, most preferably from above.

An advantage of the invention is above all that the sample does not need to be dispensed from the container to a separate vessel for analysis. Among other things, an additional advantage is that in practise the upper edge of the container and especially the upper edges between the containers of the container unit can be made thinner than the walls between the wells of the well plate. In this way a wider surface, via which the sample may be subjected to an analysis, is achieved.

The container scan be made such that they are suited for use with pipettes also used otherwise in laboratories.

Racks, which are suited for use with analysing devices also used otherwise with sample plates in laboratories, may be used.

The containers can be manufactured by injection moulding of some plastic suited for this purpose.

A number of embodiments of the invention are described in detail in the following examples.

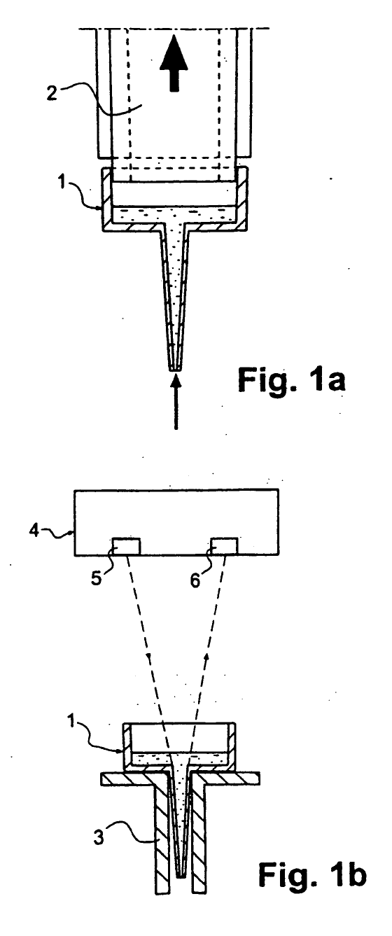

FIG. 1a illustrates a container 1, into which liquid has been sucked, connected to a suction channel of a suction device 2, such as a pipette. The container comprises a narrow conical lower container widening upwards and a wide cylindrical upper container. So much liquid is sucked into the tip that its upper surface rises to the upper part of the container. The container is dimensioned in such a way that the liquid remains in the container by means of adhesion forces, even if the suction is suspended. The inner diameter of a container made of a conventional plastic used for pipette tips (for example polyalkene) could in that case be for example from 0.5 to 1 mm. The surface of the suction channel of the suction device may comprise a vertically movable exhaust collar, by which the container can be detached from the pipette by pushing. FIG. 1b illustrates a container removed from the suction device and placed to a rack 3 in upright position. The rack comprises a recess, which supports the container. The liquid in the container can be optically analysed using an analysing device 4 from above the container. The analysing device may comprise for example a light source 5 for directing light, such as excitation light, to the sample, and a detector 6 for detecting the light, such as emission light, coming from the liquid. The rack may especially be such that it comprises several recesses. The recesses may especially be in a format corresponding to some standard well plate, thus an analysing device dimensioned to this plate may be used for examining containers placed to a rack.

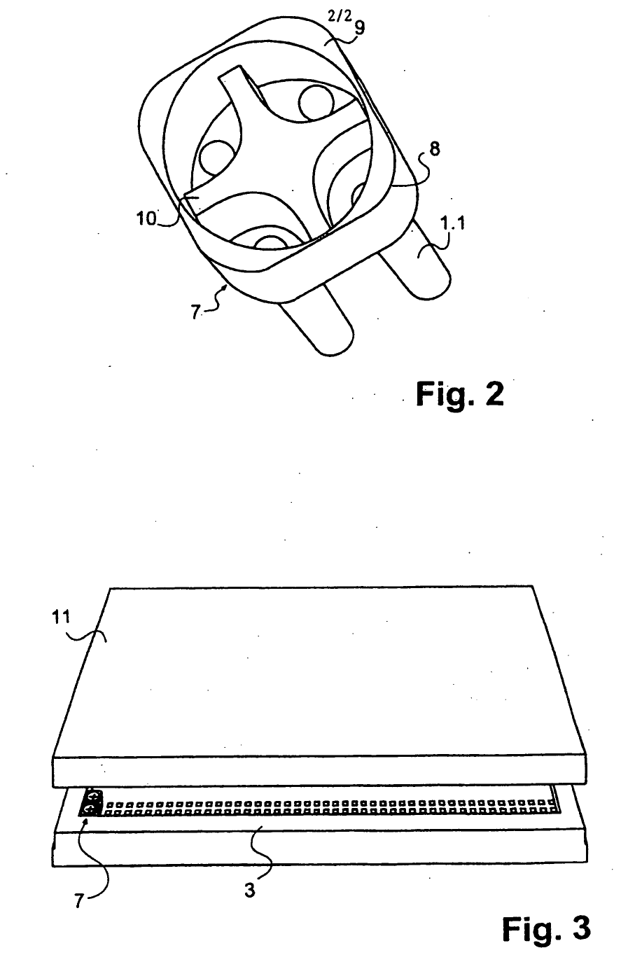

FIG. 2 illustrates a container unit 7, in which four containers 1.1 have been combined symmetrically by a common upper part 8. The upper part comprises an outer wall 9 surrounding the unit, whose inner surface is round in cross-section and by which the unit can be attached to the suction device. The interior of unit has been divided symmetrically in four sections by inner walls 10, which are lower than the outer wall so that an upper container, into which liquid can be sucked is formed inside each container. When using a dry container it is better to use stronger suction speed than when using a wetted container.

The container units 7 may be placed to a rack 3 (FIG. 3) for examination. The rack may be closed with a cover 11, whose lower surface sets tightly on the upper part of each container unit.

1-11. (canceled)

12. A container for a liquid sample, which container (1; 1.1) comprises
an elongate casing into which container the liquid sample is taken
an open lower end and an open upper end, whereby the upper end of the container is surrounded by an edge, by which it is placed in a suction device in order to suck the liquid sample into the container, after which the container is to be detached from the suction device for examining the sample in the container in the longitudinal direction of the container.

13. A container (1; 1.1) according to claim 12, which comprises a narrower lower container and a wider upper container.

14. A container (1; 1.1) according to claim 13, in which the upper container comprises a bottom.

15. A container (1; 1.1) according to claim 12, in which the container is to be detached from the suction device for examining the sample in the container in the longitudinal direction of the container from above.

16. A container (1,1) according to claim 12 combined with at least one other container (1.1) as a container unit (7).

17. A container (1.1) according to claim 16 combined so that the upper ends of the containers are surrounded by an edge (9), by which the container unit may be placed in the suction device in order to direct the suction to the containers simultaneously in order to suck liquid samples into them.

18. A rack to be used with a container (1; 1.1) according to claim 12, which rack (3) has at least one place for the container.

19. A rack according to claim 18, comprising a cover (10) forming with the rack a case, inside which the containers (1; 1.1) can be closed, most preferably so that the cover sets tightly against the upper end of each container.

20. A suction device to be used with a container (1; 1.1) according to claim 12, in which the upper end of the container is surrounded by an edge, by which it can be placed to the suction device in order to suck a liquid sample into the container.

21. A method for analysing a liquid sample, in which method the liquid sample is taken into an elongate container, in which it is examined in the longitudinal direction of the container, characterised in that the liquid sample is taken by means of a suction device into an elongate container (1; 1.1) comprising an open lower end and an open upper end, the container is detached from the suction device, and in that the sample in the container is examined optically in the longitudinal direction of the container.
